(19) United States
(12) Patent Application Publication
(10) Pub. No.: US 2021/0170092 A1
(43) Pub. Date: Jun. 10, 2021

(54) PEN NEEDLE ASSEMBLY APPARATUS

(71) Applicant: Becton, Dickinson and Company, Franklin Lakes, NJ (US)

(72) Inventors: Junyu ZHOU, Shanghai (CN); Xi ZENG, Suzhou, Jiangsu (CN); Wei HU, Shanghai (CN); Huasheng HUANG, Suzhou (CN)

(21) Appl. No.: 17/048,044

(22) PCT Filed: Apr. 12, 2019
(86) PCT No.: PCT/US2019/027126

§ 371 Date: Oct. 15, 2020

(30) Foreign Application Priority Data
Apr. 18, 2018 (CN) 201810345779.9
Apr. 18, 2018 (CN) 201820549944.8

(51) Int. Cl. A61M 5/00; A61M 5/32

(52) U.S. Cl. CPC A61M 5/002; A61M 5/008; A61M 2005/004; A61M 2005/3215; A61M 5/3213

(57) ABSTRACT

A pen needle assembly apparatus includes a tray (34), a movable clamping assembly (36) for gripping a pen needle assembly (18), and an actuator assembly (38). The tray (34) has a top end (42), a bottom end (40), and an inner cavity for storing used pen needle assemblies. The clamping assembly (36) in one embodiment includes at least one movable clamping member (48) that is movable between a first position where the clamping member engage and grip the outer cover (22) of the pen needle assembly (18) so that the needle hub (16) can be separated from the outer cover (22) and placed back in the outer cover (22) after use of the pen needle. The actuator assembly (38) is connected to the movable clamping member (48) where movement of the actuator releases the movable clamping member to move to the open positon and release the outer cover of the pen needle assembly. The base (32) of the apparatus can include an opening for engaging the inner shield (24) of the pen needle assembly to assist in removing the inner shield (24) from the needle hub (16).

### FIELD OF THE INVENTION

This application claims priority to Chinese Application No. 201810345779.9 filed Apr. 18, 2018 and Chinese Utility Model Application No. 201820549944.8 filed Apr. 18, 2018, which are incorporated by reference in their entireties.

The present invention relates generally to an apparatus for use in mounting and attaching a pen needle assembly to a delivery device, such as a pen delivery device. The apparatus has a first well or opening with a movable clamping member for retaining a needle hub having an inner shield covering a cannula on the needle hub for attaching the needle hub to the delivery device. The apparatus has a second well to receive the inner shield for separating the inner shield from the needle hub without the need to handle the inner shield. The apparatus is constructed to retain and store the needle hub after use to reduce the risk of inadvertent needle stick.

### BACKGROUND

Insulin and other injectable medications are commonly delivered with drug delivery pens. The drug delivery pen has a disposable pen needle hub attached to the pen to facilitate drug container access and allow fluid egress from the container through the needle into the patient.

Various pen needle delivery devices are known in the art for dispensing the substance to the patient. The delivery devices often use a disposable needle hub having a cannula or needle extending from a patient end of the hub for inserting into the patient. A non-patient end of the hub is coupled to the pen delivery device for delivering the substance to the patient.

The needle hub is often packaged in a container containing several pen needle hubs. A needle hub is selected from the package and attached to the pen needle delivery device for injecting the patient and then removed to be discarded. The needle hub package includes an outer cover that encloses the needle hub and a removable seal that is peeled from the outer cover to open the cavity of the cover so that the needle hub can be removed. The needle hub can have threaded non-patient end that is threaded onto the delivery device. The delivery device with the attached needle hub is then removed from the outer cover. An inner needle shield is attached to the needle hub to cover the cannula until the device is ready for use. The inner shield is removed to expose the cannula for use to deliver the substance to the patient. After use, the needle hub can be inserted back into the outer cover to enclose the exposed cannula. The pen delivery device is separated from the needle hub leaving the needle hub within the outer cover.

The prior devices require the use of both hands to connect to and remove the needle hub from the delivery device. During the placement back into the outer cover, the cannula is exposed and provides an increased risk of accidental needle stick. The manual operation of holding the outer cover while positioning the spent needle hub and cannula into the cavity of the outer cover can be difficult without accidental needle stick.

Existing pen needle assemblies are disclosed in U.S. Patent Application Publication Nos. 2006/0229562 to Marsh et al. and 2007/0149924 to R. Marsh, the entire contents of both of which are hereby incorporated by reference for this purpose.

Although the prior devices have been suitable for the intended use, there is a continuing need in the industry for improved packaging for a pen needle hub assembly.

### SUMMARY

The present invention is an apparatus and device for receiving and supporting at least one pen needle assembly for attaching the pen needle assembly to a delivery device, such as a delivery pen. In particular, the apparatus is for use with a pen needle assembly that can be used in a manner to reduce the risk of inadvertent needle stick while attaching and removing the pen needle assembly from the delivery pen. The apparatus is also able to store used pen needles for disposal.

The apparatus includes a movable clamping assembly and an actuator mechanism to operate the clamping assembly. In one embodiment, the apparatus is provided with an opening or well for a pen needle assembly. The pen needle assembly includes an outer cover, a needle hub supporting a cannula or needle, and an inner shield covering the cannula. A removable seal closes the open end of the outer cover that is removed before attaching the needle hub to the delivery pen. The apparatus retains and supports the pen needle assembly and the needle hub and cannula while attaching to a delivery pen or other delivery device. The apparatus is able to store a used needle hub and the outer cover for disposal.

The apparatus has a recess or well for receiving and retaining the inner shield that covers the cannula so that the inner shield can be removed from the needle hub without the operator handling the inner shield. The used needle hub can be inserted back into the outer cover and transferred to a storage area, tray, or internal sharps container for disposal. The inner shield optionally can be re-attached to the needle hub or can be discarded in the storage area. An actuator mechanism is provided to operate the clamping assembly and release the needle hub, outer cover, and inner shield to a suitable disposal area, bin, or container, such as a sharps container in the apparatus.

One aspect is to provide an apparatus or device for receiving and supporting a pen needle assembly to attach a needle hub to a delivery device without the need for the user to handle the needle hub with an exposed cannula on the needle hub. The needle hub assembly is positioned in the apparatus so that the delivery device can be attached to the non-patient end of the needle hub. An inner needle shield on the needle hub can then be inserted into an opening in the apparatus to grip the inner shield by an extraction mechanism so that the inner shield can be separated from the needle hub without the user handling the inner shield. After use, the needle hub is positioned in the outer cover that is retained in an opening of the apparatus and disconnected from the delivery device where the apparatus retains the used needle hub and outer cover.

The apparatus includes a housing having an internal cavity for receiving pen needles for attaching to a delivery pen and storing used pen needle assemblies and the associated components after use. The housing has a clamping assembly mechanism with at least one clamping member, such as a movable clamping member, that is able to grip the outer cover of a pen needle assembly so that the needle hub can be attached to the delivery pen. The used needle hub can be inserted back into the outer cover that is retained in the apparatus and separated from the delivery pen. An actuator can be deployed to release the outer cover so that the outer cover and used needle hub can be removed from the apparatus or deposited directly into a storage area or compartment in the apparatus for storing used pen needle assemblies until ready for disposal.

A pen needle apparatus in one aspect comprises a clamping assembly including a movable clamping member and an actuator. A tray forming a housing has an inner cavity, a top end, and a bottom end. The movable clamping member is configured for gripping an outer cover of a pen needle assembly when the movable clamping member is in a first closed position and for releasing the outer cover when the clamping member is in a second open position. The actuator is connected to the apparatus and the movable clamping member to enable the clamping member to move between the first position and the second open position to release the outer cover.

In one embodiment, the movable clamping member is spring biased to the first open position. A suitable latching mechanism engages the movable clamping member to latch the movable clamping member in the closed position. The latching mechanism is associated with the actuator to engage the movable clamping member to retain the clamping member in the closed position. The actuator is deployed to release the clamping member to enable the clamping member to move to the open position by the biasing force of the spring.

The clamping assembly in one embodiment includes at least one movable clamping member and fixed clamping member that can grip and retain the outer cover. The movable clamping member can move in a direction relative to the fixed clamping member to capture and release the outer cover. In one embodiment, the movable clamping member can slide in a substantially linear direction away from the fixed clamping member. The sliding movement can be in a direction substantially perpendicular to a center axis of an opening formed between the movable clamping member and the fixed clamping member.

The assembly includes the actuator, such as a button, that can include a mechanism to release the movable clamping member to release the outer cover. The actuator can have a connecting body with a detent that cooperates with a detent on the movable clamping member to latch or lock the movable clamping member in a closed position and release the movable clamping member to enable the movable clamping member to move to an open position. The movable clamping member and the actuator can be spring biased to a starting position. In one embodiment, the movable clamping member can be manually moved to the closed position where the movable clamping member is latched or locked in the closed position until the actuator is deployed.

The features are further provided by a pen needle assembly apparatus for receiving a pen needle assembly, where the pen needle assembly apparatus includes a base having a plurality of openings, each of the openings having a clamping assembly including a fixed clamping member oriented with a first side of each of said openings. A movable clamping member is coupled to the base on a second side of the opening, and complementing the respective fixed clamping member, where the movable clamping member is movable between an open position and a closed position to grip a pen needle assembly. IN one embodiment, the movable clamping member slides in a slot in the base in a linear direction toward and away from the fixed clamping member. An actuator is included to move the movable clamping member to the open position to release the pen needle assembly.

After use of the needle hub and cannula, the needle hub attached to the delivery pen is inserted into the outer cover that is retained in the apparatus. The needle hub can be gripped by a friction fit or interference fit to the outer cover so that the pen needle delivery device can be separated from the needle hub without the operator handling the needle hub thereby reducing the risk of inadvertent needle stick.

The features are further provided by a pen needle assembly apparatus comprising a base having a plurality of openings, where each of the openings having a clamping assembly. The clamping assembly has a fixed clamping member associated with a first side of each of the openings. The fixed clamping member has a gripping surface configured for gripping a pen needle assembly. The clamping assembly has a movable clamping member coupled to the base on a second side of each of the openings and is movable between an open position and a closed position for gripping the pen needle assembly between the fixed clamping member and the movable clamping member. The movable clamping member is biased to the open position. An actuator is movable between a first position where the movable clamping member is locked in the closed position and a second position to release said movable clamping member so that the movable clamping member moves to the open position by a spring member and releases the pen needle assembly from between the clamping members.

The features of the apparatus are further attained by a method of coupling the needle hub of a pen needle assembly to a delivery device using a pen needle assembly apparatus. The method comprises the steps of inserting a pen needle assembly into an apparatus having an opening with a dimension for receiving the pen needle assembly, where the apparatus has a fixed clamping member oriented on a first side of the opening and a movable clamping member on a second side of the opening, where the movable clamping member is movable between an open position and a closed position for gripping an outer cover of the pen needle assembly inserted into the opening; coupling the pen needle delivery device to a needle hub of the pen needle assembly; removing the pen needle delivery device and pen needle hub for delivery of a substance to a patient; returning the pen needle delivery device and pen needle hub into the outer cover and separating the pen needle delivery device from needle hub; and actuating an actuator to release the pen needle assembly from the opening in the assembly.

The objects, advantages, and salient features of the invention will become apparent from the following detailed description, which, taken in conjunction with the annexed drawings, discloses exemplary embodiments of the invention.

### BRIEF DESCRIPTION OF THE DRAWINGS

The above benefits and other advantages of the various embodiments of the present invention will be more apparent from the following detailed description of exemplary embodiments of the present invention and from the accompanying figures, in which:

FIG. 1 is an exploded perspective view of a pen needle delivery device in one embodiment showing the pen needle assembly that includes a needle hub supporting a cannula, inner shield, and outer cover;

FIG. 2 is a cross-sectional view of the pen needle assembly;

FIG. 3 is a perspective view of the apparatus and storage device in one embodiment of the invention;

FIG. 4 is a side view of the apparatus in one embodiment;

FIG. 5 is a front view of the apparatus of FIG. 3;

FIG. 6 is a top view of the apparatus;

FIG. 7 is an exploded perspective view of the apparatus;

FIG. 8 is a top view of the base of the apparatus without the clamping members and actuators;

FIG. 9 is a bottom view of the base of the apparatus of FIG. 8;

FIG. 10 is bottom view of the base of the apparatus showing the ends of the actuator buttons and the clamping members for removing the inner cover of the pen needle assembly;

FIG. 11 is a top perspective view of the fixed gripping member separated from the base;

FIG. 12 is a bottom view of the fixed gripping member of FIG. 11;

FIG. 13 is a perspective view of the apparatus showing the actuator mechanism and the collection tray removed;

FIG. 14 is a bottom perspective view of the movable clamping member of the gripping mechanism;

FIG. 15 is a top perspective side view of the movable clamping member of FIG. 14;

FIG. 16 is a top perspective view of the body of the ratchet member of the movable clamping member;

FIG. 17 is a top perspective view showing a actuator button connected to the body of the ratchet member;

FIG. 18 is a top perspective view of the actuator button;

FIG. 19 is a bottom perspective view of the actuator button of FIG. 18;

FIG. 20 is a top perspective view of the connecting arm and detent of the actuating mechanism;

FIG. 21 is a perspective view of the gripping spring member;

FIG. 22 is a perspective view of the movable clamping member showing the spring member for gripping the pen needle hub;

FIG. 23 is a partial cross-sectional view showing the actuating mechanism;

FIG. 24 is a top perspective view of the apparatus showing the movable clamping member in the closed position;

FIG. 25 is a partial top view of the apparatus showing the movable clamping member in the closed position;

FIG. 26 is a partial cross-sectional view of the actuator mechanism showing the movable clamping member in the open position;

FIG. 27 is a cross-sectional view showing the movable clamping member in the open position;

FIG. 28 is a perspective view showing the actuator mechanism retaining the movable clamping member in the open position;

FIG. 29 is a cross sectional view of the actuator mechanism;

FIG. 30 is a top view of the apparatus showing the movable clamping member in the closed position;

FIG. 31 is a bottom perspective view of the apparatus showing the movable clamping member in the closed position; and

FIG. 32 is a partial cross-sectional view showing the movable clamping members in the closed position.

Throughout the drawings, like reference numbers will be understood to refer to like parts, components, and structures.

### DETAILED DESCRIPTION OF THE EXEMPLARY EMBODIMENTS

The present invention is directed to a pen needle apparatus for storing and supporting at least one and preferably a plurality of pen needle assemblies for use with a pen needle delivery device. The apparatus can function as a storage device for a used needle hub where the used needle hub can be replaced in the apparatus after use and later discarded without handling the needle hub to reduce the risk of accidental needle stick.

Reference are made in detail to embodiments of the present invention, which are illustrated in the accompanying drawings, wherein like reference numerals refer to like elements throughout. The embodiments described herein exemplify, but do not limit, the present invention by referring to the drawings. The exemplary embodiments are presented in separate descriptions, although the individual features and construction of these embodiments can be combined in any number of ways to meet the therapeutic needs of the user.

This disclosure is not limited in its application to the details of construction and the arrangement of components set forth in the following description or illustrated in the drawings. The embodiments herein are capable of being modified, practiced or carried out in various ways. The phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not limited to physical or mechanical connections or couplings. Further, terms such as up, down, bottom, and top are relative, and are employed to aid illustration, but are not limiting. The features of the assembly can be combined with other embodiments so long as they do not contradict each other. The term “substantially” mean mostly or entirely and can include a range or value. The term can include a given value or a reasonable range encompassing the value. Terms of degree, such as “substantially” or “approximately” are understood by those skilled in the art to refer to reasonable ranges around and including a given value.

The apparatus for the needle hub assemblies can enable the convenient and easy assembly and removal of the needle hub on the pen delivery device with reduced handling of the exposed needle cannula. The device for the needle hub assemblies can be placed on a flat surface, such as a table, so that the pen needle delivery device can be attached to the needle hub assembly with one hand thereby reducing the risk of the needle stick by handling the needle hub with the exposed needle cannula.

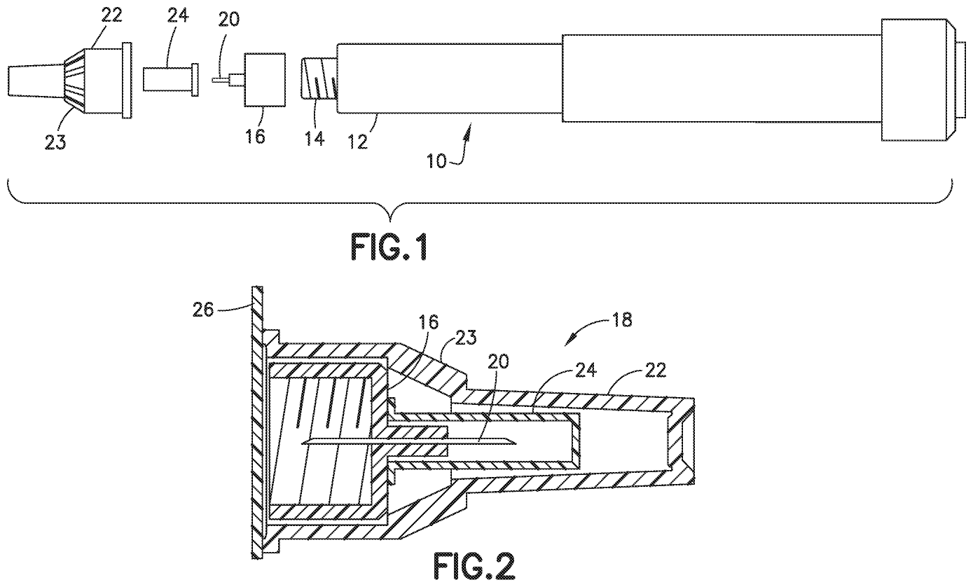

Pen needle delivery device 10, as shown in FIG. 1 typically comprises a dose knob/button, an outer sleeve 12, and a cap. A dose knob/button allows a user to set the dosage of medication to be injected. The outer sleeve 12 is gripped by the user when injecting medication. The cap is used by the user to securely hold the pen needle device 10 in a shirt pocket or other suitable location and provide cover/protection from accidental needle injury.

In standard pen needle devices the dosing and delivery mechanisms are all found within the outer sleeve 12 and is not described in greater detail here as they are understood by those knowledgeable of the delivery device. A medicament cartridge is typically attached to a standard pen injector housing by known attachment mechanism. The distal movement of a plunger or stopper within the medicament cartridge causes medication to be forced into the reservoir housing. The medicament cartridge is sealed by a septum and punctured by a septum penetrating needle cannula located within a reservoir or housing. Reservoir housing preferably is screwed onto the medicament cartridge although other attachment means can be used. The pen needle delivery device can be a standard pen delivery device known in the industry so that the pen needle delivery device is not shown in detail. The pen needle assembly 18 as shown in FIG. 2 includes a needle hub 16 supporting a cannula 20, an outer cover 22, and an inner shield 24. A protective seal 26 is attached to the open end of the outer cover as shown in FIG. 2 to enclose the needle hub and cannula to maintain a clean and sterile condition. The seal 26 can be a label or other closure member that can be peeled easily from the outer cover to access the needle hub during use.

The pen needle delivery device 10 is connected to needle hub 16 shown in FIG. 1 that has a connecting non-patient end with internal threads that screw onto a threaded end 18 of the delivery device 10. The needle cannula 20 extends from the patient end of the needle hub 16 for delivering the substance to the patient. The outer cover 22 can be provided to cover the needle cannula to protect the patient from accidental needle stick before and after use. The outer cover 22 includes ribs 23 to assist in gripping the outer cover during use. The inner shield 24 is provided over a post extending from the end of the needle hub 16 to enclose the cannula. During use, the needle hub 16 is connected to the pen delivery device and the inner shield is removed. After use, the outer cover is generally placed back on needle hub to cover the needle cannula. The needle hub with the cover is then removed from the pen needle delivery device and discarded.

The apparatus 30 is configured for receiving and supporting at least one pen needle assembly 18 in a position where the delivery device 10 can be coupled to the needle hub 16 for use for injecting the substance to the patient without the need for the user to handle the needle hub 16 with the exposed cannula. The used needle hub 16 and cannula 20 can then be positioned back into the outer cover 22 that is retained in the apparatus and separated from the delivery device. The used outer cover and needle hub and the inner shield are retained in the assembly. In the embodiment shown, the apparatus is configured for receiving a plurality of needle assemblies.

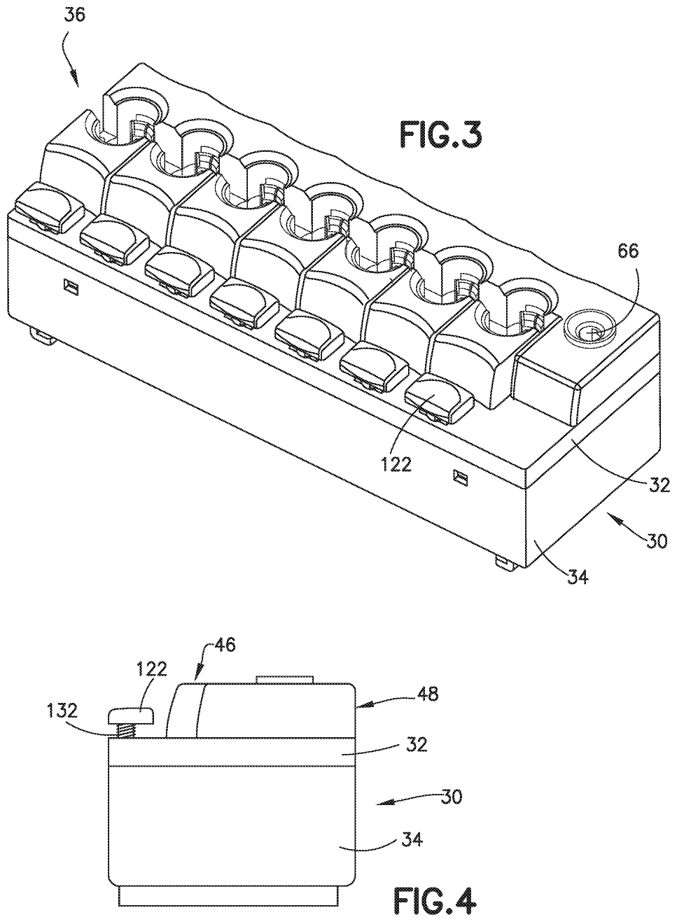

An apparatus 30 is provided for receiving a pen needle assembly 18 for coupling the pen needle hub 16 to the pen needle delivery device and for separating the pen needle hub from the pen needle delivery after use. In one embodiment, the apparatus 30 includes a base 32 coupled to a housing 34 forming a tray, a retaining and clamping assembly 36, and an actuator assembly 38. The housing 34 as shown in FIG. 3 has a top end 40 and a bottom end 42. Bottom end 42 can have a bottom face with a non-slip surface. The nonslip surface can be molded directly onto the bottom face or can be a nonslip material, such as an elastomeric material or resilient polymer, applied to the bottom surface to retain the housing in a stable position during use on a table or other flat surface.

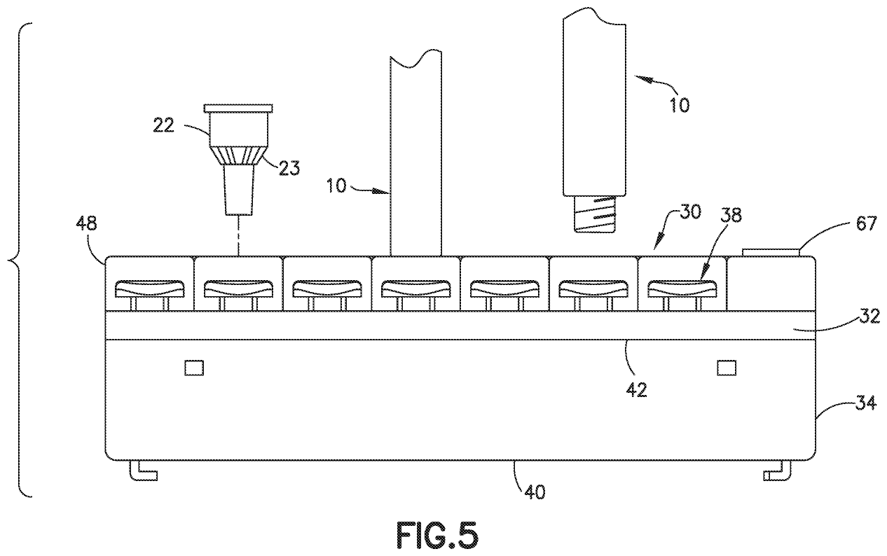

As shown in the drawings the apparatus 30 is configured for receiving a plurality of pen needle assemblies at the same time. The apparatus 30 includes a plurality of clamping assemblies 36 with a respective actuator assembly 38 positioned adjacent each other where each clamping assembly 36 and actuator assembly 38 are independently operable. Each clamping assembly 36 of the apparatus 30 is able to support a pen needle assembly until ready for use and discard the pen needle assembly individually after use. A pen needle delivery device can be connected to the respective pen needle assembly and stored in the apparatus 30 until ready for use as shown in FIG. 5. At that time, the pen needle delivery device and a single needle hub are removed for use.

The tray 34 has an inner cavity with a dimension sufficient to retain and store a plurality of used pen needle assemblies. In the embodiment shown, a housing 34 is formed to couple with the base 32 by hooks 44 extending from the base 32 for mating with a complementing tab or hook within the tray 34. As shown in the drawings, the base 32 defines a removable cover for the tray 34.

Referring to FIGS. 3-6, the clamping assembly 36 is provided for supporting and gripping a pen needle assembly for connecting the needle hub 16 to the delivery pen and disconnecting the needle hub from the delivery pen after the use. The clamping assembly 36 is a movable assembly that can move between an open position for receiving the outer cover of the pen needle assembly and a closed position for engaging and gripping the outer cover the pen needle assembly.

In the embodiment shown, the clamping assembly 36 includes two opposing clamping members or jaws where at least one is movable relative to the other. In one embodiment the clamping assembly 36 includes a fixed clamping member 46 coupled to the base 32 and a movable clamping member 48 that is movable relative to the base 32 and the fixed clamping member 46. The base 32 has an opening 76 with a dimension complementing the outer dimension of the pen needle assembly so that the pen needle assembly can slide through into the housing when released by the clamping assembly 36. As shown, an opening 76 is provided for each clamping assembly 36.

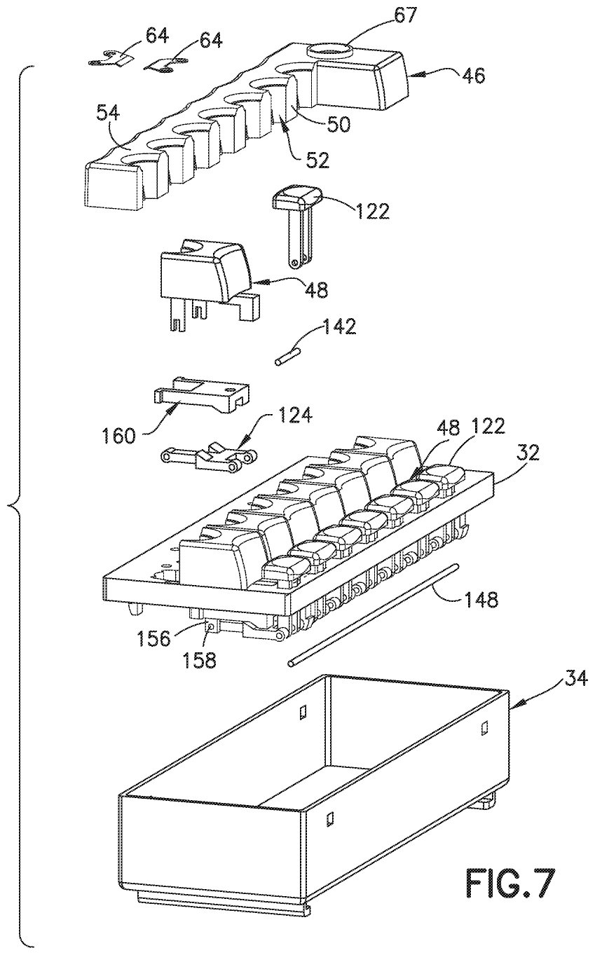

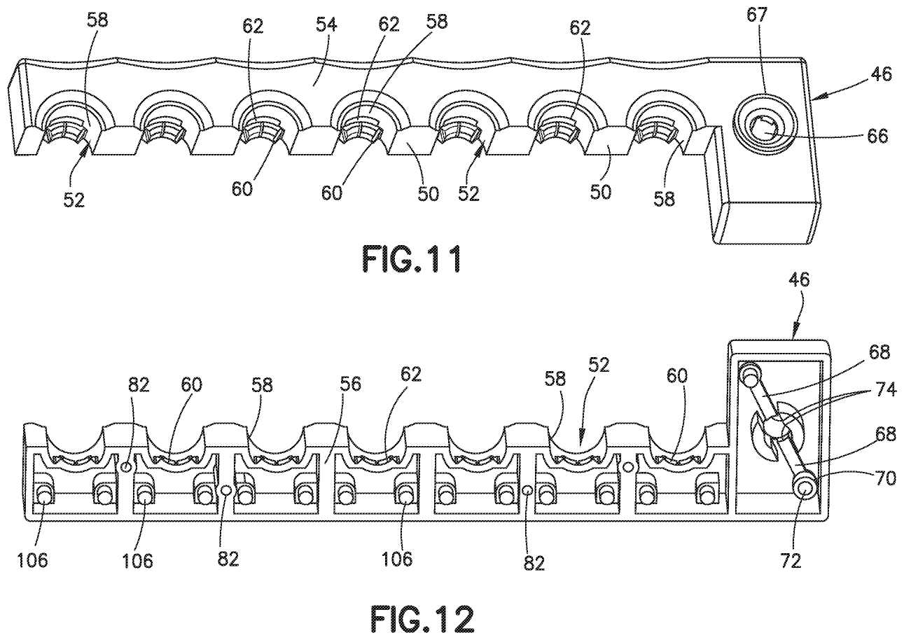

The fixed clamping member 46 in the embodiment shown is formed as a separate member that is attached to the base 32 by a suitable fastening mechanism such as adhesives or mechanical fasteners. As shown in FIGS. 7, 11 and 12, the fixed clamping member 46 has a front face 50 with a plurality of recessed areas 52 extending between a top face 54 and a bottom face 56. In the embodiment shown, the front face 50 is substantially flat for mating with the movable clamping member 48. As shown in FIG. 11, the recessed areas 52 are spaced apart from each other and have a substantially U-shape inner surface 58 with a dimension and configuration to accommodate the pen needle assembly 18. The inner surface 58 in the embodiment shown is provided has an arcuate shaped flange 60 that is able to grip the outer surface of the pen needle assembly. A slot 62 is formed in the inner surface 58 to receive a spring metal slip gripping member 64 as shown in FIG. 21.

An extractor mechanism can be provided in the base or the fixed clamping member for separating the inner shield from the needle hub without the need for the technician to manually remove the inner shield. Referring to FIGS. 11 and 12, an aperture 66 is formed at one end of the fixed clamping member 46 and has a dimension to receive the inner shield 24 of the pen needle assembly. Alternatively, the aperture 66 is formed in eh top face of the base 32. A collar 67 extends from the top face 54 and surrounds the aperture 66. A gripping member shown as the spring metal clip 68 is attached to the bottom surface of the fixed clamping member 46 next to the aperture 66. The spring metal clip 68 has a first end 70 attached to a boss 72 extending from the bottom face 56 of the fixed clamping member 46 and a second free end 74 extending a slight distance into the opening defined by the aperture 66 a distance sufficient to engage the inner shield.

In the embodiment shown, the two spring metal clips 68 are oriented on opposite sides of the aperture 66 where the free ends 74 extend into the opening of the aperture a distance sufficient to grip the outer surface of the inner shield 24. During use, the inner shield 24 is manually pushed through the aperture 66 where the spring clips 68 grips the outer edge of the inner shield with a sufficient force to enable needle hub to separate from the inner shield by pulling needle hub 16 upward while retaining the inner shield within the aperture. During subsequent use, a new inner shield is pushed downwardly against the inner shield captured by the spring clips 68 to push the original inner shield through the aperture to be deposited into the tray 34 below the aperture 66.

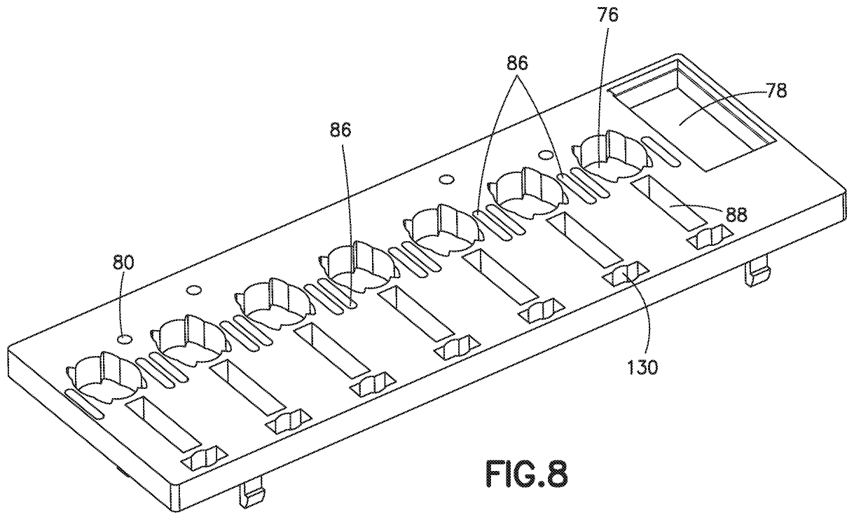

Base 32 has a dimension for mating with the open top end of the housing 4 to define a tray or enclosure for receiving the used pen needle assemblies. Referring to FIGS. 7-9, base 32 has a substantially rectangular configuration with a plurality of openings 76 associated with each of the fixed clamping members 46 and movable clamping members 48. FIG. 8 is a top view of the base 32 showing the opening 76 aligned next to each other and an opening 78 at one end cooperating with the aperture 66 of the fixed clamping member 46 to enable the used inner shield to fall into the tray 34. A plurality of apertures 80 are provided on the base 32 to receive the corresponding boss 82 on the bottom side of the fixed clamping member 46 for coupling the fixed clamping member 46 to the base 32 as shown in FIG. 12.

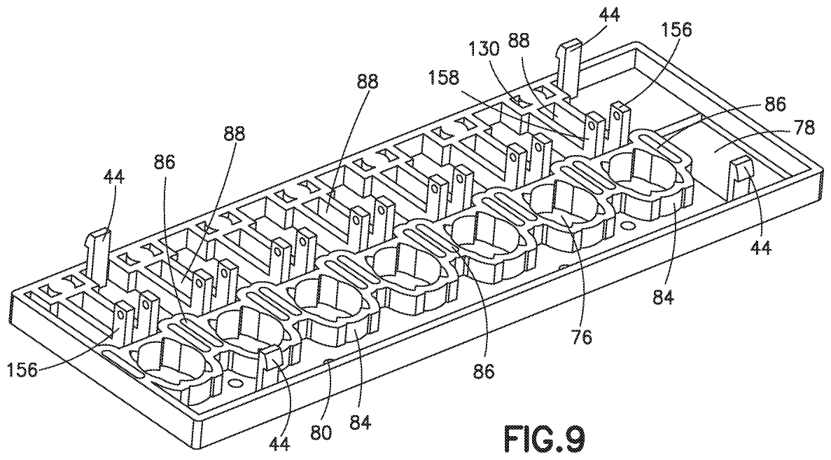

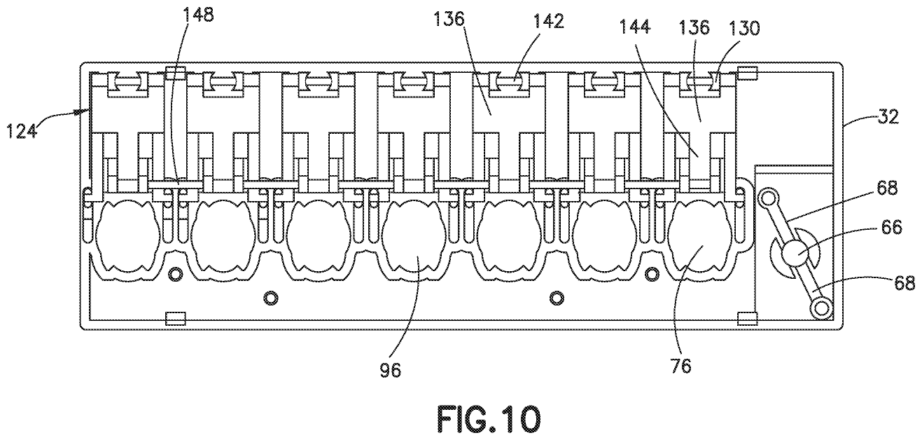

FIG. 9 is a bottom view of the base 32 showing the openings 76 surrounded by a collar 84. Each opening 76 includes a pair of guide slots 86 for cooperating with the movable clamping member 48 and allowing the moving clamping member 48 to slide in a linear direction perpendicular to a center axis of the opening 76. An elongated slot 88 extends away from the respective openings 76 for cooperating with the movable clamping member 48.

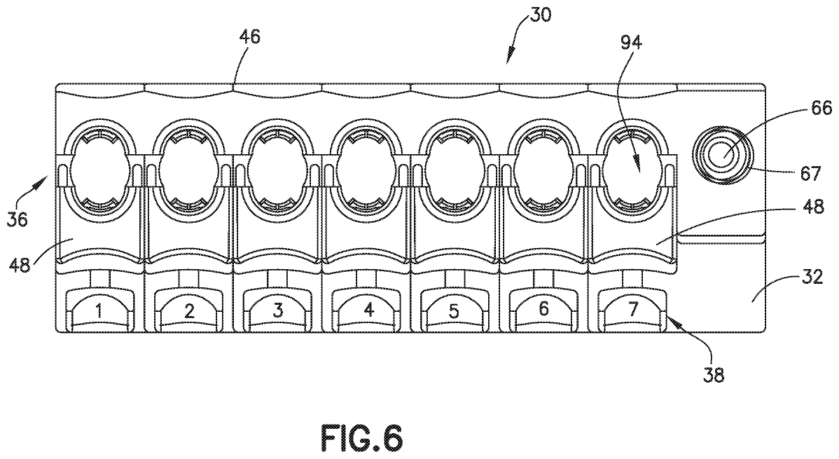

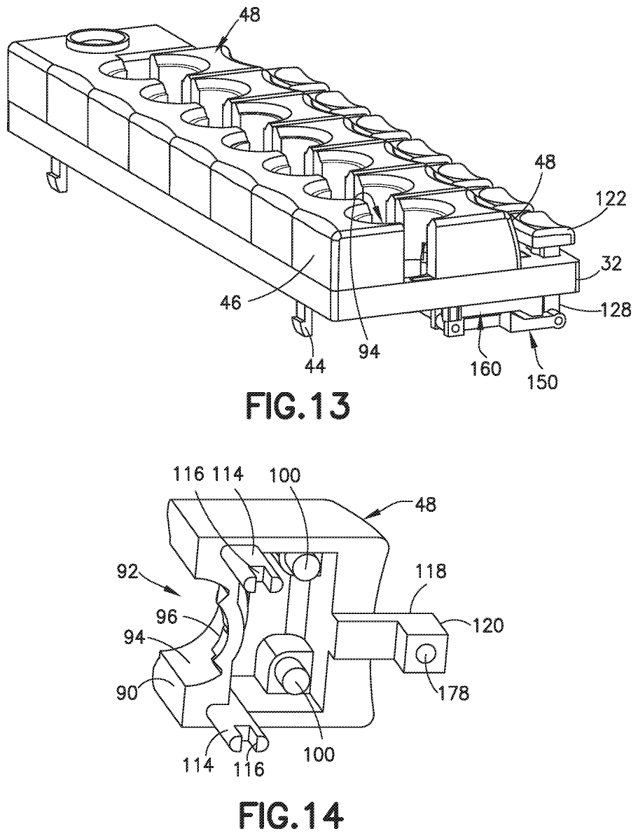

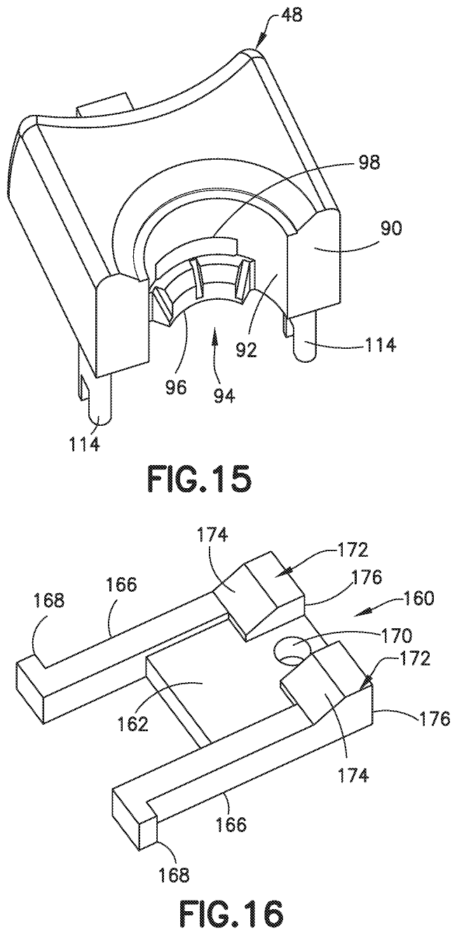

Referring to FIGS. 14 and 15 the movable clamping member 48 has a front face 90 for mating with the front face 50 of the fixed clamping member 46. A recessed area 92 has a substantially U-shape to complement the U-shaped recess 52 of the fixed clamping member 46 to form a well 94 or opening for receiving and clamping the pen needle assembly as shown in FIG. 6 and FIG. 13. As shown FIG. 15, the U-shaped recess 92 includes an angled flange 96 for gripping the pen needle assembly. A slot 98 in the recess 92 receives a metal spring clip 64 shown in FIG. 21 forming a gripping member.

As shown in FIG. 14 the bottom face of the clamping member 48 includes spaced apart posts 100. The posts 100 are positioned for attaching the spring metal clip gripping member 64 to the movable clamping member 48. As shown in FIG. 21, the spring metal clip 64 includes a pair of legs 102 having an aperture 104 for mating with posts 100 of the clamping member 48 and corresponding posts 106 on the bottom side of the fixed clamping member 46 as shown in FIG. 12. The spring metal clip 64 has an inclined end portion 108 forming a gripping edge 110 that extends through the slot 98 to engage the pen needle assembly. As shown in FIG. 21 the gripping edge 110 as a concave recessed area 112 for mating with the outer surface of the pen needle assembly. The inclined end portion 108 is angled downwardly with respect to the well 94 or opening to enable the outer cover to slide past the gripping edge 110 while resisting removal of the outer cover in an upward direction.

Referring to FIG. 14 the movable clamping member 48 includes two spaced apart posts 114 having a length to extend through the guide slots 86 of the base 32. The distal ends of the post 114 include a notch 116 for mating with the actuator assembly 38. A leg 118 extends from the movable clamping member 48 in a direction opposite the recessed area 92. The distal end of the leg 118 includes a downwardly extending tab 120 extending through the slot 86 for mating with the actuator assembly 38.

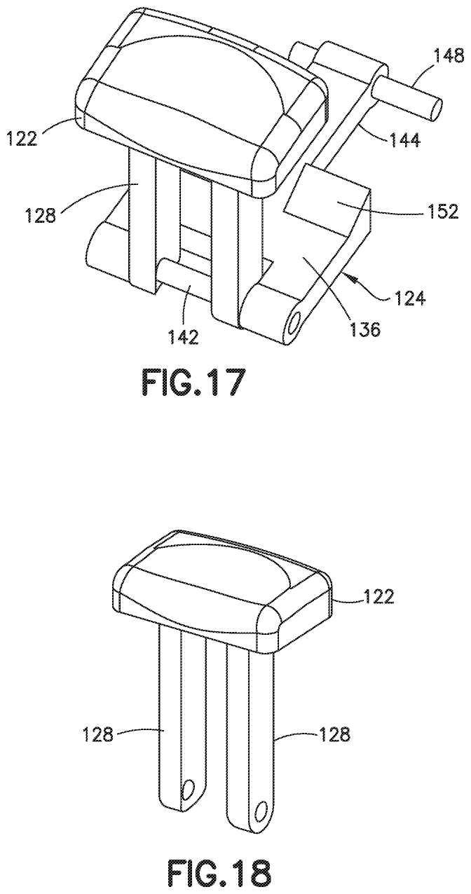

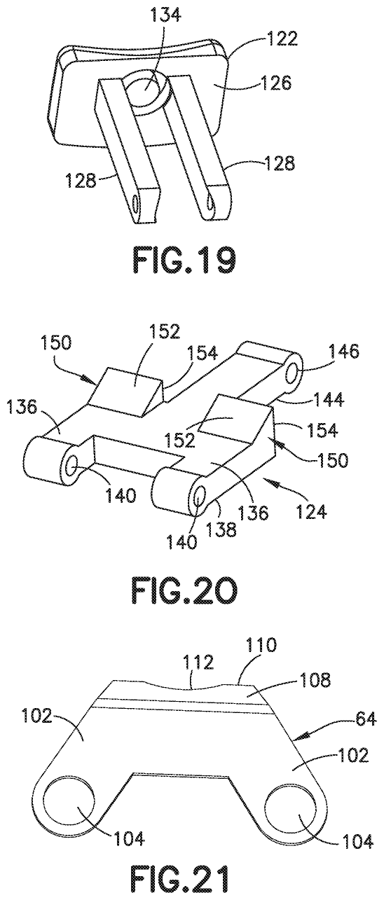

The actuator assembly 38 in the embodiment shown includes an actuator button 122 and a ratchet assembly 124 shown in FIG. 17. As shown in FIG. 18, actuator button 122 includes a top portion and legs 128 that pass through the openings 130 in the base 32 to enable the actuator button 122 to reciprocate in an up-and-down direction relative to the base. In the embodiment shown, two legs 128 are provided although a single leg can be used. In one embodiment a spring 132 is positioned between the base 132 and the actuator button 122 for biasing the actuator button 122 in an upward direction away from the base as shown in FIG. 23. As shown in FIG. 19 a post 134 is provided in the bottom face of the actuator button 122 for mating with and positioning the spring 132 on the actuator button 122 between the legs 128.

Ratchet assembly 124 as shown in FIG. 20 includes a ratchet connecting member having a body 136 with legs 138 extending from one end. In the embodiment shown, two parallel legs 138 are provided although a single or multiple legs can be used. The distal end of each leg 138 includes an aperture 140 for receiving a pivot pin 142 for connecting the legs 138 to the legs 128 of the respective actuator button 122 as shown in FIG. 17. The opposite end of the body 136 includes a leg 144 having an aperture 146 for receiving a pivot pin 148 for pivotally connecting the body 136 to the base 32. As shown in FIG. 20, body 136 includes at least one detent 150. In the embodiment shown, two spaced apart detents are provided for mating with the movable clamping member 48. In the embodiment shown, detents 150 have an inclined face 152 forming a cam surface and an opposite flat face 154 oriented substantially perpendicular to the longitudinal dimension of the body 136. Referring to FIG. 9 the bottom face of the base 32 includes two raised posts 156 at one end of the respective guide slots 86. Each post 156 includes an aperture 158 for receiving a pivot pin 142 for pivotally connecting the leg 144 of the body 136 to the base 32 as shown in FIGS. 7 and 23.

A complementing ratchet member 160 as shown FIG. 16 and FIG. 21 is attached to the bottom side of the movable clamping member 148. In the embodiment shown, the ratchet member 160 is fixed to the movable clamping member 160. Referring to FIG. 16, ratchet member 160 includes a body 162 having legs 166 extending from one end. In the embodiment shown, two spaced apart and parallel legs 166 are provided. Each leg 166 has an outwardly extending tab 168 for coupling with the respective movable clamping member 48. The opposite end of the body 162 includes an aperture 170 for mating with the aperture 170 on the movable clamping member 48.

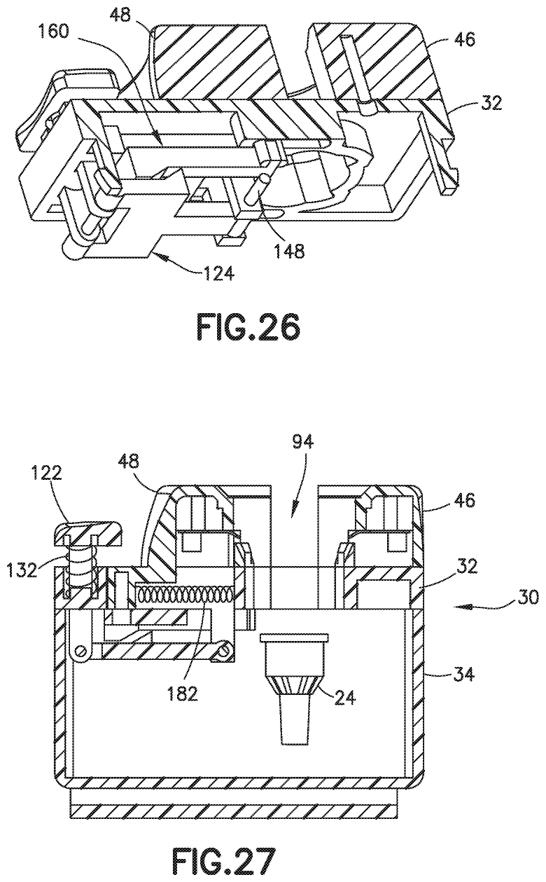

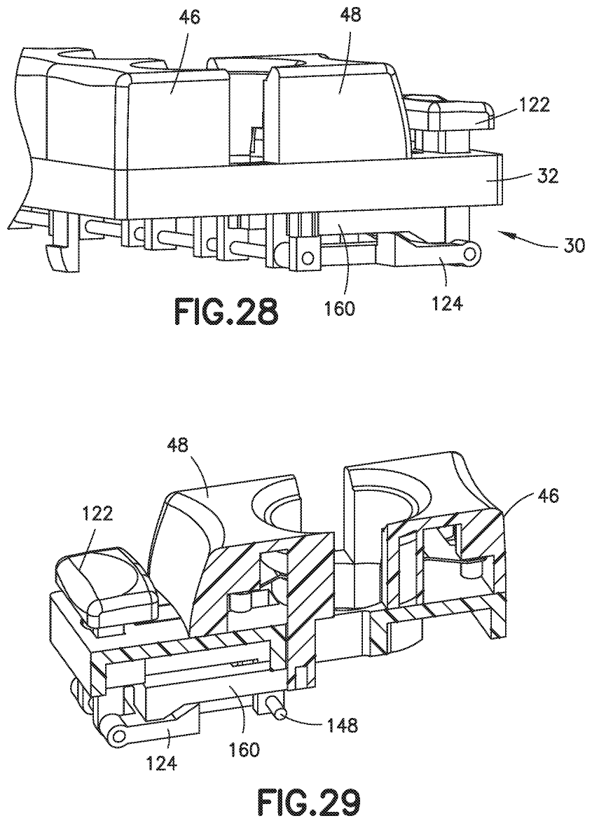

Detents 172 are provided on opposite sides of the aperture as shown in FIG. 16. The detents 172 have an inclined face 174 forming a cam surface and a flat face 176 extending substantially perpendicular to a longitudinal dimension of the body 62. As shown in FIG. 26 ratchet member 160 is coupled to the movable clamping member 48 by the tabs 168 mating with the notch 116 formed in the posts 114 of the movable clamping member 48. The movable clamping member is positioned on the outer top face of the base 32 with the legs 114 extending through the apertures in the base. The ratchet member 160 is positioned below the apertures in the base. The ratchet member 160 is positioned below the base 32 opposite the movable clamping member and attached to the movable clamping member so that the movable clamping member and ratchet member move a single unit in a linear direction. A fastener, such as a screw, can extend through the aperture 170 formed in the body 162 for mating with an aperture 178 formed in the bottom face of the tab 120.

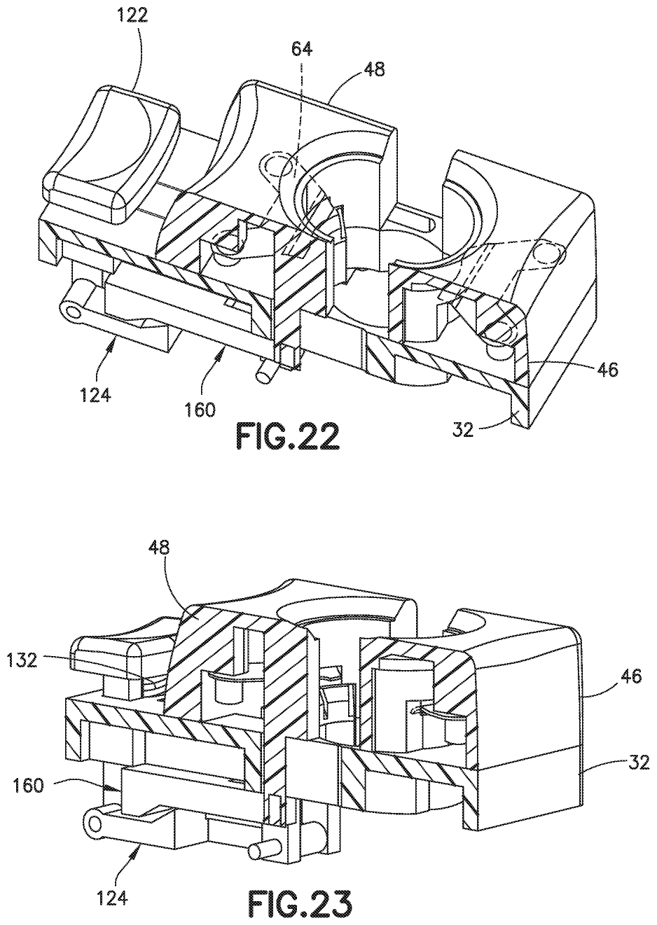

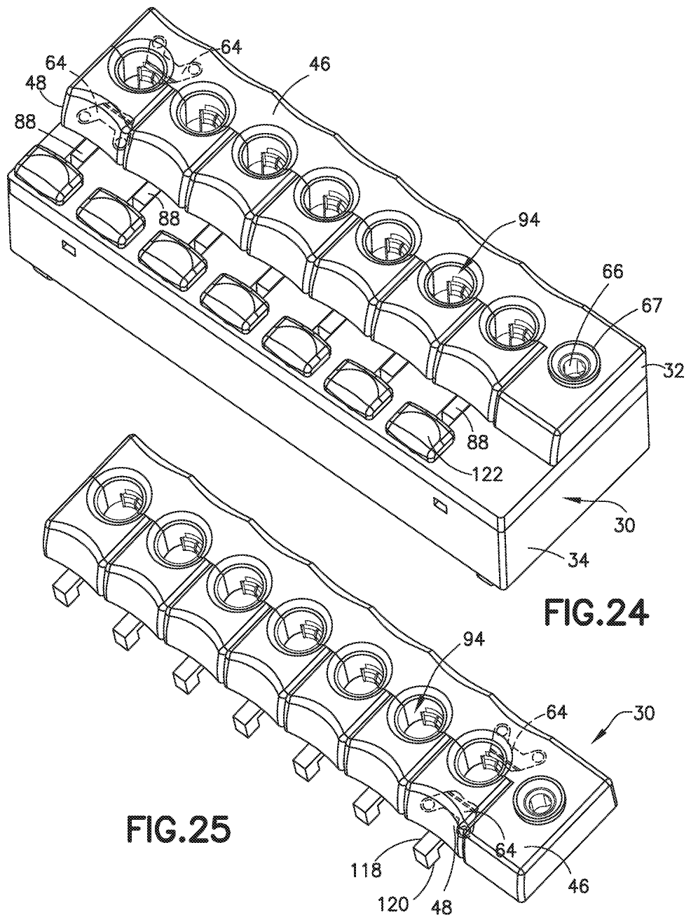

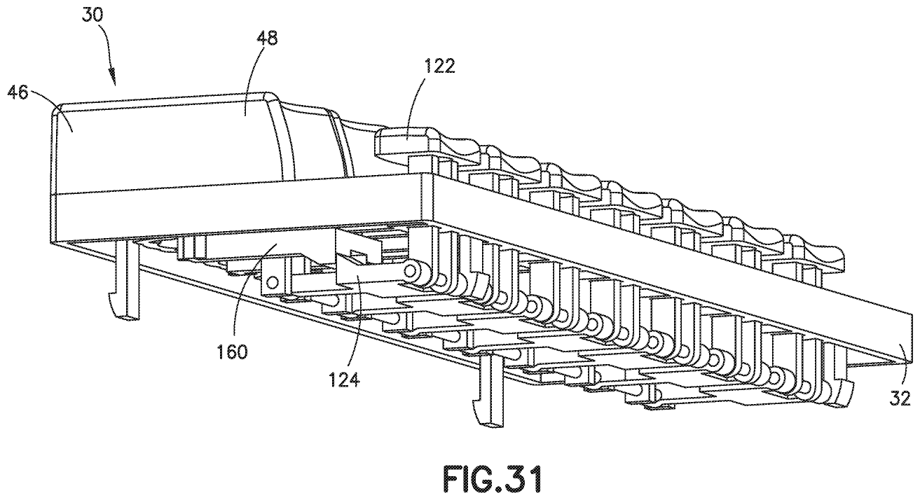

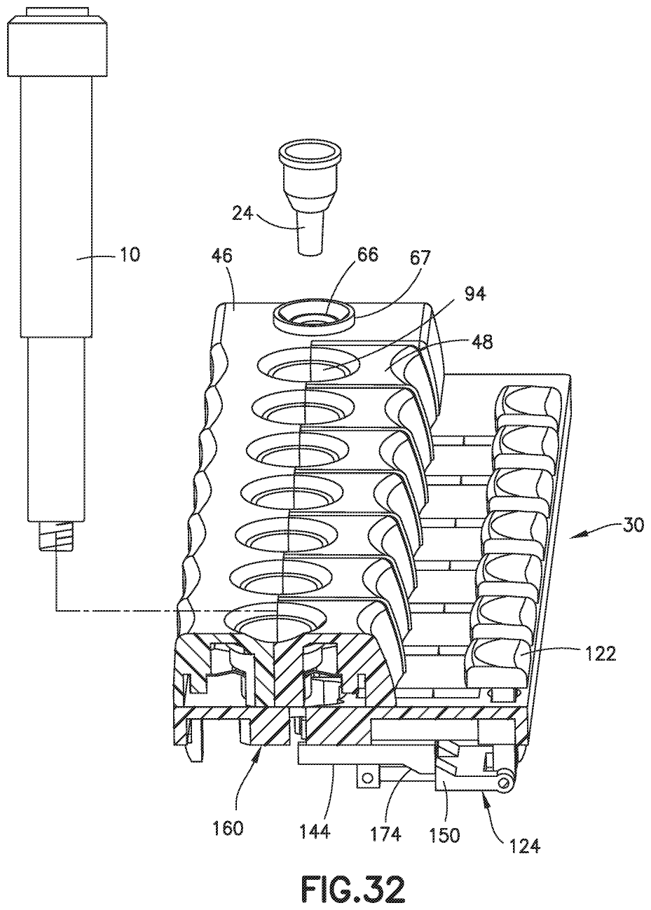

In the embodiment shown, two spaced apart detents 172 are provided for mating with a respective detent 150 on the body 136 of the ratchet assembly 124. It will be understood that the number of detents can vary although the number of detents 150 of the body 136 generally correspond to the number of detents 172 of the ratchet member 160. As shown in FIGS. 22, 23, 26, and 27 the inclined face 174 of the detents 172 of the ratchet member 160 mate with the inclined face 152 of the detent 150 of the body 136 to be able to slide over each other. The movable lamping member slide manually where the inclined faces 174 of the ratchet member slide over the inclined faces 152 where the body 136 pivots about the axis of the pivot pin 148 away from the base. Once the inclined faces slide over one another, the body 136 springs back to the original position. The flat face 176 of the ratchet member 160 engages the flat face 154 of the body 136 to hold the movable clamping member 48 in the closed position as shown in FIGS. 31 and 32 until released by the actuator mechanism.

As shown in FIG. 26 and FIG. 27, the posts 114 and the leg 118 of the movable clamping member 48 extend through the respective guide slots 86 and elongated slot 88 in the base 32. The body 136 of the ratchet assembly 124 is coupled to the respective posts 114 and leg 118 to capture base 32 between the movable clamping member 148 and the body 136 and couple the movable clamping member 48 to the base 32 while allowing the movable clamping member 48 to slide in a linear direction relative to the guide slots 86 and the elongated slot 88 and relative to the fixed clamping member 46.

The legs 128 of the button 122 extend through the respective openings 130 formed in the base 32 as shown in FIG. 26 and FIG. 27. The legs 138 of the body 136 of the ratchet assembly 124 are pivotally connected to the legs 128 of the actuator button 122 by the pivot pin 142 to capture the actuator button 122 to the base 32. The leg 144 of the body 136 is connected to the respective post 156 on the bottom face of the base 32 by the pivot pin 148. As shown, the actuator button 122 slides in an up-and-down direction and is biased upward by the spring 132. The downward movement of the actuator button 122 pivots the body 136 in a downward direction relative to the base 32.

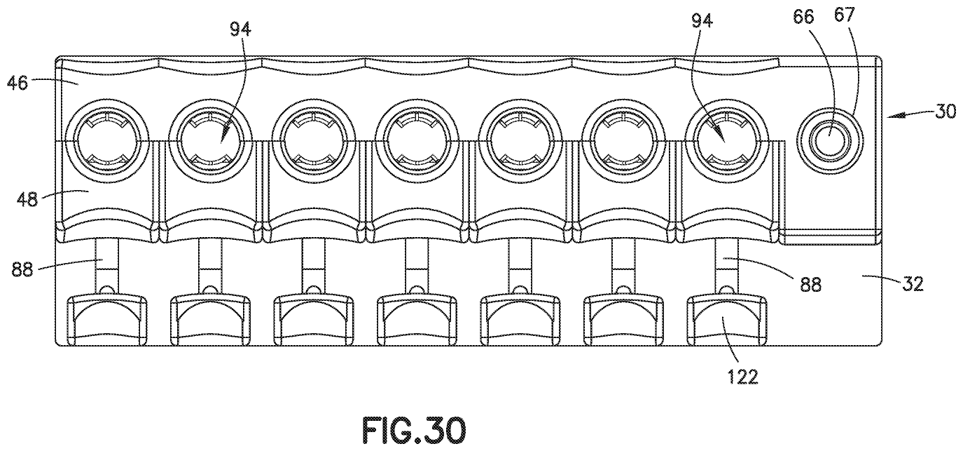

As shown in FIG. 27 a spring 182 is positioned in the longitudinal slot 88 and extends between the post 120 and the inner edge of the slot 88 to bias the movable clamping member 48 to the position shown in FIG. 27 and the open position relative to the fixed clamping member 46. During use, the movable clamping member 48 is pushed manually toward the fixed clamping member 46 against the biasing force of the spring 182 to the position shown in FIGS. 30 and 32. As shown in FIG. 27 inclined surface 174 of the ratchet member 160 attached to the movable clamping member 48 opposes the inclined face 152 of the detent 150 on the body 136. The movable clamping member 48 can slide towards the fixed clamping member 46 so that the inclined surfaces 152 and 174 are able to slide over each other by pivoting the body 136 downward relative to the pivot pin 142 to separate the detents allowing the movable clamping member 48 to slide toward the fixed clamping member 46. Once the inclined surface 174 slide past the inclined surface 152, the biasing force of the spring 132 returns the body 136 to the original position to latch or lock the movable clamping member 48 in the closed position as shown in FIGS. 31 and 32 by the face 176 of the ratchet member 160 of FIG. 16 engaging the flat face 154 of the ratchet assembly 124 of FIG. 20. Manually sliding the movable clamping member 48 to the closed position relative to the fixed clamping member 46 as shown in FIG. 32 enables the flat face 176 of the detent 172 to engage the flat face 154 of the detent 150 to capture and latch the movable clamping member in the closed position and resist separation of the movable clamping member 48 relative to the fixed clamping member 46.

Once the movable clamping member 48 is latched in the closed position with respect to the fixed clamping member 46 shown in FIG. 23, the apparatus can be used for coupling a pen needle assembly 18 to a delivery device such as a pen needle 10. A pen needle assembly 18 can be positioned in one or more of the wells 94 formed by the closed clamping members 46 and 48. The pen needle assembly 18 can be manually placed in the respective well 94 or opening and pushed downwardly until the flanges 60 and 96 and spring clip 64 engage the outer cover 22. Typically the outer cover 22 is secured when an audible sound or tactile click provides an indication of a proper connection of the pen needle assembly to the well 94.

The flanges 60 and spring metal clip 64 are able to grip the outer surface of the outer cover 22 to resist rotation of the outer cover relative to the clamping members and resist removal of the outer cover from the well 94. The label 26 can then be peeled from the outer cover 22 to expose the needle hub 16 for use. The delivery device such as the pen needle 10 is then inserted into the needle hub 16 and threaded onto the needle hub in the usual manner. The delivery device with the needle hub 16 attached is removed from the outer cover 22 to expose the inner shield 24. The inner shield 24 is then inserted through the aperture 66 to engage the spring clip 68. The delivery device is pulled away to separate the inner shield 24 from the needle hub 16. The inner shield 24 can be pushed manually through the opening in the base 32 into the housing 34 for disposal. Alternatively, a subsequent inner shield can be pushed into the aperture 66 pushing the previous inner shield through the opening into the housing 34.

The delivery device with the needle hub attached can be prepared for use by the technician such as by adjusting the dose and expelling air from the delivery device and cannula 20. At this stage, the needle hub 16 and the delivery device can be inserted back into the respective outer cover 22 until ready for use. A delivery device can be prepared for each pen needle assembly positioned in a respective well 94. The respective actuator button 122 can include indicia such as a number or letter to designate a selected patient, dosage or medication contained in the delivery device. Each of the wells formed by the clamping members 46 and 48 receive a pen needle assembly for preparing a delivery device for use. In this manner, a plurality of delivery devices can be prepared simultaneously for use where each delivery device is positioned in a respective well identified by the indicia on the actuator button.

Each of the delivery devices can be used to deliver the medication to the patient. After use, the needle hub 16 while still attached to the delivery device is inserted back into the respective outer cover 22 where the delivery device can be separated from the respective needle hub and leaving the used needle hub in the cover retained in the well formed by the clamping members 46 and 48. The respective actuator button 122 for the respective well 94 can then be depressed causing the body 136 of the ratchet mechanism 124 to pivot away to separate the detent 150 on the body 136 from the detent 172 on the body 166 of the ratchet member attached to the movable clamping member 48. The spring 182 then biases the movable clamping member 48 to the open position allowing the used pen needle assembly to separate from the clamping member 46 and 48 and drop into an area in the tray 34 for safe disposal. The movable clamping member 48 can then be manually pushed to the closed position for receiving a new pen needle assembly. In this manner, the used pen needle assemblies can be discarded into the tray after use without the need for the technician to handle the needle hub thereby reducing the risk of needle stick during use and disposal of the delivery device and the needle hub.

In another embodiment, the extractor can be formed from a sheet metal clip attached to the base where the clip includes sharp edges to engage the inner shield. The extractor can have a substantially keyhole shaped slot with an enlarged, substantially circular opening and a longitudinally extending opening having a width less than the diameter of the circular opening. The circular opening can have a dimension slightly larger than the outermost dimension of the inner shield 24 so that the inner shield can pass through without interference. The distal end of the longitudinal opening can include at least one tooth projecting inwardly with respect to the circular opening. The tooth can have an inclined surface forming a sharp tip and inclined in a generally downward direction to allow the inner shield 24 to slide over the tip and resist removal of the inner shield in an upward direction. The tooth can be sufficiently flexible so that the inner shield can be pushed through the opening to pass into the tray or other receptacle in the housing.

The steps can be repeated by placing a new pen needle assembly in the housing and the delivery pen can be attached to the needle hub. The new needle hub with the inner shield 24 can be inserted into the opening for the extractor where the tip of the inner shield engages the top end of the previous shield so that a downward force pushes the previous shield past the flexible edges of the spring clip. The inner shield 24 is then able to drop downward into the tray.

In a further embodiment shown in FIG. 21 and FIG. 22 the outer cover 22 can be re-attached to the hub 16 without separating the needle hub from the delivery pen. The delivery pen with the attached needle hub 16 can be inserted through the collar and the housing so that the needle hub 16 is received in the outer cover 22. The actuator 108 can then be depressed to separate the clamping members to release the outer cover 22. The delivery pen can then be withdrawn with the outer cover protecting the cannula to avoid inadvertent needle stick.

The foregoing embodiments and advantages are exemplary and are not to be construed as limiting the scope of the present invention. The description of an exemplary embodiment of the present invention is intended to be illustrative, and not to limit the scope of the present invention. Various modifications, alternatives, and variations will be apparent to those of ordinary skill in the art, and are intended to fall within the scope of the invention. It is particularly noted that the features of different embodiments and claims may be combined with each other as long as they do not contradict each other. Accordingly, all such modifications are intended to be included within the scope of this invention as defined in the appended claims and their equivalents.

1. A pen needle apparatus comprising:
a base having an opening, a clamping assembly including a fixed clamping member coupled to said base, and a movable clamping member, said fixed clamping member oriented with a first side of said opening, said movable clamping member coupled to said base on a second side of said opening, said movable clamping member complementing said fixed clamping member for receiving a pen needle assembly between the clamping members, said movable clamping member being movable between an open position and a closed position to grip a pen needle assembly; and
an actuator mechanism to move said movable clamping member to the open position to release the pen needle assembly.

2. The pen needle apparatus of claim 1, wherein said movable clamping member is spring biased to the open position.

3. The pen needle apparatus of claim 2, wherein said actuator mechanism includes an actuator that is movable between a first position and a second position and includes a ratchet member to retain said movable clamping member in the closed position when said actuator is in the first position, and said actuator being movable to the second position to release said movable clamping member where said movable clamping member moves to the closed position.

4. The pen needle apparatus of claim 3, wherein said ratchet member includes a first detent for engaging a second detent on said movable clamping member to retain said movable clamping member in the closed position.

5. The pen needle apparatus of claim 4, wherein said actuator comprises a movable button coupled to said ratchet, and where moving said button to a deployed position disengages said first detent on said ratchet member from said second detent of said movable clamping member.

6. The pen needle apparatus of claim 5, wherein said ratchet member has a body with a first end pivotally connected to said base to pivot about a fixed pivot point relative to said base, and a second end pivotally connected to said button, where said body includes said first detent for engaging the second detent of the movable clamping member.

7. The pen needle apparatus of claim 1, wherein said apparatus includes a collection area position below said base, and where said pen needle assembly falls into said collection area when released by said movable clamping member.

8. The pen needle apparatus of claim 1, wherein said movable clamping member is spring biased to the open position, and said actuator in a first position retains said movable clamping member in the closed position, and said actuator is movable to a second position to disengage from said movable clamping member whereby said movable clamping member is spring biased to said open position.

9. The pen needle apparatus of claim 8, wherein said apparatus includes a housing with an open end, said base coupled to said open end of said housing, and said opening in said base is oriented with said open end of said housing whereby the pen needle passes through said opening when said movable clamping member moves to the open position.

10. The pen needle apparatus of claim 8, wherein said movable clamping member is manually moved from said open position to said closed position.

11. The pen needle apparatus of claim 10, wherein said actuator comprises a latching mechanism to engage said movable clamping member to retain said movable clamping member in the closed position, and where said latching mechanism is movable to a position to disengage from said movable clamping member to spring bias said movable clamping member to the open position.

12. The pen needle apparatus of claim 11, wherein said latching mechanism comprises a ratchet member having one end pivotally coupled to said base and being spring biased toward said base to a first position, said ratchet having a detent for engaging said movable clamping member when said movable clamping member is in the closed position and said ratchet member is in the first position.

13. The pen needle apparatus of claim 12, wherein said ratchet member is movable to a second position to disengage from said movable clamping member.

14. The pen needle apparatus of claim 1, wherein said base has a plurality of openings, each of said openings having a respective fixed clamping member, a respective movable clamping member, and a respective actuator, each movable clamping member and actuator being independently operable.

15. A pen needle apparatus comprising:
a base having a plurality of openings, each of said openings has a fixed clamping member associated with a first side of a respective opening, said fixed clamping member having a gripping surface configured for gripping a pen needle assembly;
a plurality of movable clamping members, each clamping member coupled to said base on a second side of a respective opening and being movable between an open position spaced from said fixed clamping member and a closed position for gripping the pen needle assembly between the fixed clamping member and the movable clamping member, said movable clamping member being biased to the open position; and
an actuator associated with each of a respective movable clamping member and fixed clamping member, said actuator movable between a first position to retain the respective movable clamping member in the closed position and a second position to release said movable clamping member where said movable clamping member is biased to the open position to release the pen needle assembly.

16. The pen needle assembly apparatus of claim 15, further comprising a spring member engaging said movable clamping member to bias the movable clamping member to the open position.

17. The pen needle assembly apparatus of claim 16, wherein said actuator includes a ratchet member to engage a detent on the movable clamping member to retain said movable clamping member in the closed position.

18. The pen needle apparatus of claim 17, wherein said ratchet member is movable to a positon to disengage from said movable clamping member to bias said movable clamping member to the open position.

19. The pen needle assembly apparatus of claim 16, wherein said actuator includes a body having a first end pivotally connected to the base to define a fixed pivot point of said body, and a second end pivotally connected to the actuator.

20. The pen needle assembly apparatus of claim 19, wherein said body includes a detent oriented between said first end and said second end for releasably engaging a detent on the movable clamping member.

21. A pen needle assembly apparatus of claim 20, wherein said actuator is spring biased to the first position where said detent on said body engages said detent on said movable clamping member, and where said actuator is movable to the second position to disengage said detent on said body from the detent on the movable clamping member whereby said movable clamping member is biased to the open position.

22. A method of assembling a pen needle assembly to a delivery device comprising:
inserting a pen needle assembly into an apparatus having an opening with a dimension for receiving the pen needle assembly, where said apparatus has a fixed clamping member oriented on a first side of the opening and a movable clamping member on a second side of the opening, where said movable clamping member is movable between an open position and a closed position for gripping an outer cover of the pen needle assembly inserted into the opening;
coupling the delivery device to a needle hub of the pen needle assembly;
removing the delivery device and pen needle hub for delivery of a substance to a patient;
returning the delivery device and pen needle hub into the outer cover and separating the pen needle delivery device from needle hub; and
actuating an actuator to release the pen needle assembly from the opening in the assembly.

23. The method of claim 22, wherein said apparatus includes a plurality of openings for receiving a respective pen needle assembly, each said opening having a fixed clamping member, a movable clamping member, and an actuator, said method comprising inserting a pen needle assembly into each opening for coupling to a respective delivery device, and selectively actuating the actuator to release pen needle assembly after use.

24. The method of claim 23, wherein said apparatus includes a tray positioned below said openings, and said method comprising actuating a selected actuator to release and deposit a used pen needle assembly into said tray.
